(19) United States
(12) Patent Application Publication
(10) Pub. No.: US 2003/0124097 A1
(43) Pub. Date: Jul. 3, 2003

(54) Method to improve the safety of gene therapy

(76) Inventors: Richard F. Potter, Denfield (CA); Sarah D. McCarter, London (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/310,991

(22) Filed: Dec. 6, 2002

Related U.S. Application Data
(60) Provisional application No. 60/336,700, filed on Dec. 7, 2001.

(51) Int. Cl. A61K 48/00

(52) U.S. Cl. 424/93.2; 514/44

(57) ABSTRACT

Novel methods for improving gene therapy protocols are provided. The method involves administering a nucleic acid construct, preferably a viral vector, comprising an anti-inflammatory protein to an animal receiving gene therapy. The anti-inflammatory protein is preferably a heme oxygenase.

This application claims the benefit under 35 USC §119(e) from U.S. Provisional patent application serial No. 60/336,700 filed Dec. 7, 2001.

### FIELD OF THE INVENTION

The present invention relates to methods and compositions for improving gene therapy.

### BACKGROUND OF THE INVENTION

Human trials and animal experiments have demonstrated that the replication-defective adenovirus vector has high potential for use in gene therapy. Adenovirus vectors can be produced inexpensively at high titres and are highly effective at transferring exogenous genes to both replicating and non-replicating cells. Following infection, the transferred adenovirus genome remains epichromosomal, resulting in no risk of insertional mutagenesis. However, the use of adenovirus vectors in gene therapy has been limited by immune responses in the target organ, causing transient gene expression and the inability to re-administer the adenovirus (1,2). It has been demonstrated that the immune responses to adenovirus vectors are biphasic in nature (3-8). An early non-specific acute inflammatory response occurs over the first 4 days following adenovirus administration. A late specific acquired immune response begins 5 to 7 days post infection and lasts for several weeks. Until recently, research focussed on the late phase, which is directed against viral antigens and is characterized by antibody formation and the infiltration of cytotoxic T-lymphocytes (9-10). The early phase is now believed to be the most important determinant of the efficiency of in vivo gene transfer and expression (11). Studies have shown that acute inflammation is responsible for the loss of 70-90% of the transferred genes (12) and is directly linked to early tissue injury (13). Perhaps the most convincing evidence of the importance of controlling this acute inflammation is the disturbing result of the 1999 clinical trial where acute inflammation resulted in the death of one volunteer receiving liver-directed gene therapy (14-15).

The acute inflammation elicited by adenovirus vectors is characterized by early neutrophil infiltration, followed by monocyte and macrophage accumulation. In the lung, neutrophil accumulation was found as early as 6 hours following administration (11) and peaked 2 days after injection (5,6,16) with mononuclear infiltrates on the third day (11). Neutrophil accumulation in salivary glands was most evident 1-2 days after infection with predominantly mononuclear infiltrates on the third day (7). In the liver, significant infiltrates of neutrophils, monocytes, macrophages, and natural killer cells, are visible as early as 1 hour following infection (13). The acute inflammatory response is due to direct toxicity of the virus, is multiplicity-of-infection dependent and is present in immunodeficient animals (6,7,11,12). This inflammation also appears to be unrelated to virus-based gene expression (7), is independent of the adenoviral backbone (17) and is independent of both the type of expression cassette and particle-to-pfu ratio of the vector preparation (12). Whether this acute inflammation is dose-dependent is controversial (12,13).

Heme oxygenase (HO) is the rate-limiting enzyme in the degradation of heme to carbon monoxide (a vasodilator), iron which is converted into ferritin (an antioxidant), and biliverdin which is subsequently converted into bilirubin (an antioxidant) (18,19). There are three known isozymes of HO. HO-1 is the inducible form of HO and is constitutively expressed in liver and spleen. HO-1 is a heat shock protein (HSP32) and is highly inducible by a variety of stimuli, such as heat shock, ischemia, radiation, hypoxia, hyperoxia, inflammation, and disease states. In addition, heme oxygenase has been shown to provide protection to a variety of tissues (including heart, lung, liver, brain, and kidney) following stress. Adenovirus mediated gene transfer of HO-1 has been successfully demonstrated both in vitro and in vivo. Gene transfer of HO-1 protected retinal pigment cells from hemoglobin toxicity and hemorrhage (20) and protected ocular tissues against oxidative stress (21). Gene transfer of HO-1 was used as a pretreatment to protect against hyperoxia-induced lung injury (22) and to protect liver from ischemia/reperfusion injury (23).

Although it is generally accepted that some method must be found to overcome the problem associated with the use of adenoviral transfection, to date there have been no advances in the art. There is a need in the art to provide improved methods for reducing the immune response to vectors used in gene therapy.

### SUMMARY OF THE INVENTION

The present inventors have determined that administering a viral vector containing a gene encoding a heme oxygenase prevents the acute inflammatory responses normally associated with the administration of viral vectors.

Accordingly, the present invention provides a method for reducing an immune response to a first nucleic acid construct comprising administering an effective amount of a second nucleic acid construct comprising a nucleic acid sequence encoding an anti-inflammatory protein to a cell or animal in need thereof.

Preferably, the first and second nucleic acid constructs are in a viral vector such as an adenovirus. The first and second nucleic acid constructs may be contained in the same viral vector or may be administered in separate viral vectors. When administered in separate vectors, the second vector can be administered concurrently or at some point during the treatment with the first vector.

The anti-inflammatory protein is preferably a heme oxygenase.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows representative RT-PCR results (A) and densitometric quantification (B and C) demonstrate the upregulation of HO-1 following injection of Ad-HO-1 or Ad-HO-1+Ad-β-Gal and the upregulation of β-Galactosidase following injection of Ad-β-Gal or Ad-HO-1+Ad-β-Gal.

FIG. 2 shows immunohistochemical and H&E staining of serial sections. Serial sections of liver were stained with hematoxylin and eosin (top row) or stained for HO-1 (bottom row). Representative sections 72 hours following intraperitoneal injection of vehicle (A and B), Ad-HO-1 (C and D), Ad-β-Gal (E and F), and Ad-HO-1+Ad-β-Gal (G and H) show effective gene transfer to hepatocytes by intraperitoneal injection.

FIG. 3 shows the stillframe image of an intravital video microscopy video illustrates the identification of rolling and adhered leukocytes. Three rolling leukocytes are visible in this image and indicated by arrows.

FIG. 4 is a bar graph showing the total number of leukocytes in venules following injection of vehicle, Ad-HO-1, or Ad-HO-1+Ad-β-Gal is significantly lower than the number following injection of Ad-β-Gal. This figure shows that the elevation in the number of leukocytes rolling and adhered in postsinusoidal venules by Ad-β-Gal is completely removed by concurrent administration of Ad-HO-1 at 24 and 72 hours post injection. * denotes significantly different (P<0.01) than all other groups at equivalent time.

FIG. 5 is a bar graph showing the total number of leukocytes in sinusoids following injection of vehicle, Ad-HO-1, or Ad-HO-1+Ad-β-Gal is significantly lower than the number following injection of Ad-β-Gal. This figure shows that the elevation in the number of leukocytes adhered in hepatic sinusoids by Ad-β-Gal is completely removed by concurrent administration of Ad-HO-1 at 24 and 72 hours post injection. * denotes significantly different (P<0.01) than all other groups at equivalent time.

FIG. 6 is a bar graph showing the number of leukocytes and macrophages following injection of vehicle, Ad-HO-1, or Ad-HO-1+Ad-β-Gal is significantly lower than the number following injection of Ad-β-Gal. This figure shows that the elevation in the number of leukocytes and macrophages by Ad-β-Gal is completely removed by concurrent administration of Ad-HO-1 at 24 and 72 hours post injection. * denotes significantly different (P<0.01) than all other groups at equivalent time. # denotes significantly different (P<0.05) than all other groups at equivalent time.

### DETAILED DESCRIPTION OF THE INVENTION

As hereinbefore mentioned, the present inventors have demonstrated that administering an adenovirus comprising a heme oxygenous gene reduces the inflammatory response to a second adenovirus. The invention has important implications for improving gene therapy protocols as approximately 90% of DNA transferred by adenovirus is lost during the first 24 hours following administration due to the immediate inflammatory immune response that is generated.

Accordingly, the present invention provides a method for reducing an immune response to a first nucleic acid construct comprising administering an effective amount of a second nucleic acid construct comprising a nucleic acid sequence encoding an anti-inflammatory protein to a cell or animal in need thereof.

The term “effective amount” as used herein means an amount effective, at dosages and for periods of time necessary to achieve the desired result.

The term “animal” as used herein includes all members of the animal kingdom, including humans. Preferably, the animal to be treated is a human.

The term “reducing an immune response” means that the immune response observed to the first nucleic acid construct in the presence of the second nucleic acid construct is reduced or lower as compared to the immune response observed to the first nucleic acid construct in the absence of the second nucleic acid construct. When the first and second nucleic acid constructs are in the same construct or vector then “reducing an immune response” means that the immune response generated to the vector containing the first and second nucleic acid constructs is lower than the immune response generated to a vector containing only the first nucleic acid construct. One skilled in the art can readily determine whether or not an immune response is reduced using techniques known in the art including, but not limited to, measuring a cytotoxic T cell response and/or measuring cytokine levels such as the Th1 cytokines interleukin-2 (IL-2) and interferon-γ (IFN-γ).

The term “immune response” includes both non-specific and specific immune responses. The latter term includes both cell mediated and humoral immune responses. The immune response that is reduced is preferably a non-specific immune response. More preferably, an inflammatory response. The inflammatory response can have any cause including, but not limited to, an inflammatory response caused by the viral proteins (such as the viral capsid) or the heterologous gene (such as a therapeutic gene) or both. There can also be inflammation in the patient arising from whatever disease state is being treated. One skilled in the art can readily determine whether or not an inflammatory response is reduced using techniques known in the art including, but not limited to, measuring leucocyte or macrophage levels and/or measuring proinflammatory protein or gene levels (such as tumour necrosis factor-α (TNF-α), interferon inducible protein-10 (IP-10), interleukin-6 (IL-6), interleukin-8 (IL-8), interleukin-12 (IL-12), nuclear factor-κB (NF-κB)) and/or measuring adhesion molecule levels (such as intercellular adhesion molecule-1 (ICAM-1), vascular cell adhesion molecule-1 (VCAM-1) and adhesion molecule CD34).

The anti-inflammatory protein can be any protein that can reduce an immune response to the first nucleic acid construct. Preferably, the anti-inflammatory protein is a heme oxygenase (HO). Other anti-inflammatory agents that may be used include, but are not limited to, super oxide dismutase, catalase, glutathione, nitric oxide synthase, heat shock proteins such as heat shock protein-70 or any of the anti-inflammatory steroids or anti-inflammatory cytokines such as interleukin-4 (IL-4) or interleukin-10 (IL-10).

The term “heme oxygenase” as used herein means a protein or enzyme having the activity of a heme oxygenase in that it can catalyze the conversion of heme to biliverdin, releasing iron and carbon monoxide. The term includes full length heme oxygenases as well as biologically active fragments or variants thereof. Any isoform of a heme oxygenase (HO) may be used including HO-1, HO-2 and HO-3. The sequences of these are known in the art and they can readily be used in the present invention (49-51).

In a specific embodiment, the heme oxygenase is heme oxygenase-1 (HO-1). The nucleic acid and protein sequence of HO-1 is known in the art and can be obtained from an 1.0 kbp XhoI-HindIII fragment from a rat HO-1 cDNA clone pRHO-1 (52) containing the entire coding region. As the homology for HO is maintained across species this could also apply to human HO-1 (hHO-1).

The first nucleic acid construct and second nucleic acid construct may be contained in a viral or non-viral vector, preferably a viral vector. Examples of viral vectors that may be used according to the invention includes adenoviruses, adeno-associated viruses, retroviruses, lentiviruses, vaccinia viruses and herpes virus. Most preferably, the viral vector is an adenovirus vector.

The first and second nucleic acid constructs can be present in the same viral vector or may be present in different vectors.

The first nucleic acid construct may additionally comprise a foreign or heterologous gene or nucleic acid sequence. The heterologous gene or nucleic acid sequence can encode any protein or peptide that one wishes to transfer to a recipient animal or cell including, but not limited to, therapeutic, diagnostic and/or prophylactic proteins. The second nucleic acid construct (containing the anti-inflammatory gene) will reduce the immune or inflammatory response to the heterologous gene and/or to the vector that carries the gene.

In a specific embodiment, the heterologous gene encodes a protein that is used in gene therapy. In such an embodiment, the present invention provides a method for reducing an inflammatory or immune response resulting from the use of viral vectors used for gene therapy. To this effect, a viral vector containing the anti-inflammatory agent would be administered either concurrently or during the course of treatment with a vector containing the gene construct of interest. Alternatively, the nucleic acid construct for the anti-inflammatory agent (such as heme oxygenase) may be contained within the viral vector containing the gene construct of interest. The viral vectors can be delivered either simultaneously, or separately by any route of administration targeting any cell, tissue or organ system.

Accordingly, in one embodiment, the present invention provides a method of reducing an inflammatory response to a vector used in gene therapy comprising administering (i) a first vector for gene therapy comprising a heterologous gene and (ii) a second vector comprising a gene encoding an anti-inflammatory protein to an animal undergoing gene therapy. In another embodiment, the present invention provides a method of reducing an inflammatory response to a vector used in gene therapy comprising administering a vector for gene therapy comprising a heterologous gene and a gene encoding an anti-inflammatory protein to an animal undergoing gene therapy.

As a result, the invention can be used to improve the safety and efficacy of gene therapy protocols. Examples of types of gene therapy that may be improved by the present invention include, but are not limited to, liver disease, cancer, transplantation, cystic fibrosis, nervous system disorders, genetic disorders and any gene replacement therapy.

The nucleic acid constructs or vectors of the invention will additionally include suitable regulatory sequences containing the necessary elements for expression (transcription and translation) of the anti-inflammatory protein and heterologous gene, when present. Suitable transcription and translation elements may be derived from a variety of sources including bacteria, fungi, viral, mammalian or insect genes.

In the Example described herein, a 1.0 kbp XhoI-HindIII fragment from rat HO-1 cDNA clone pRHO-1 (52) containing the entire coding region was cloned using plasmid pAC-CMVpLpA and the recombinant HO-1 adenovirus generated by recombination in either 911 or 293 N3S cells after co-transfection with the pAC-HO-1 recombinant plasmid.

Selection of appropriate transcription and translation elements is dependent on the target cell chosen, and may be readily accomplished by one of ordinary skill in the art. Examples of such elements include: a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, and sequences conferring inducibility of transcription may be incorporated into the expression vector. It will also be appreciated that the necessary transcriptional and translation elements may be supplied by the native anti-inflammatory gene (such as heme oxygenase) and/or their flanking regions.

The nucleic acid molecules may also contain a reporter gene which facilitates the selection of transformed or transfected host cells. Examples of reporter genes are genes encoding a protein such as β-galactosidase, chloramphenicol acetyltransferase, firefly luciferase, or an immunoglobulin or portion thereof such as the Fc portion of an immunoglobulin preferably IgG. Transcription of the reporter gene is monitored by changes in the concentration of the reporter protein such as β-galactosidase, chloramphenicol acetyltransferase, or firefly luciferase. This makes it possible to visualize and assay for expression of the anti-inflammatory protein.

The nucleic acid constructs or vectors can be introduced into target cells by various methods including, but not limited to, transformation, transfection, infection, electroporation, microinjection, lipofection, protoplast fusion, calcium phosphate, strontium phosphate and DEAE dextran. Methods for the transformation and transfection of host cells to express foreign DNA are well known in the art (see, e.g., Itakura et al., U.S. Pat. No. 4,704,362; Hinnen et al., PNAS USA 75:19291933, 1978; Murray et al., U.S. Pat. No. 4,801,542; Upshall et al., U.S. Pat. No. 4,935,349; Hagen et al., U.S. Pat. No. 4,784,950; Axel et al., U.S. Pat. No. 4,399,216; Goeddel et al., U.S. Pat. No. 4,766,075; and Sambrook et al. Molecular Cloning A Laboratory Manual, 2nd edition, Cold Spring Harbor Laboratory Press, 1989, all of which are incorporated herein by reference).

Suitable expression vectors for directing expression in mammalian cells generally include a promoter, as well as other transcriptional and translational control sequences. Common promoters include SV40, MMTV, metallothionein-1, adenovirus Ela, CmV, immediate early, immunoglobulin heavy chain promoter and enhancer, and RSV-LTR. Protocols for the transfection of mammalian cells are well known to those of ordinary skill in the art.

The nucleic acid constructs or vectors of the invention may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals.

The pharmaceutical composition may be administered in a convenient manner such as by injection (subcutaneous, intravenous, etc.), oral administration, inhalation, transdermal application, or rectal administration. Depending on the route of administration, the nucleic acid constructs or vectors may be coated in a material to protect the compound from the action of enzymes, acids and other natural conditions which may inactivate the compound. The preferred route of administration may include any of those mentioned above and the choice of route depends on the cell, tissue or organ system that are to be transfected.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985), Handbook of Pharmaceutical Excipients (edited by Arthur Kibbe, American Pharmaceutical Association, Washington, D.C. (2000) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456.

The following non-limiting examples are illustrative of the present invention:

### EXAMPLE 1

Methods

Adenovirus Vectors The recombinant adenovirus containing rat HO-1 cDNA has been described previously (22). Briefly, a 1.0 kbp XhoI-HindIII fragment from the rat HO-1 cDNA clone pRHO-1 (43), containing the entire coding region, was cloned into plasmid pAC-CMVpLpA (44). Recombinant HO-1 adenovirus Ad-HO-1 was generated by homologous recombination in 293 N3S cells after co-transfection with the pAC-HO-1 recombinant plasmid. The recombinant adenovirus containing the Escherichia coli β-Galactosidase gene, Ad-β-Gal, has been described (45). Briefly, the β-Galactosidase gene was cloned into the Bcg/II restriction site of the adenovirus shuttle plasmid pAdCMVlink (46). Recombinant β-Galactosidase adenovirus Ad-β-Gal was generated by homologous recombination in 293 cells after co-transfection with replication-defective sub360 viral DNA.

Animals Male C57BL6 mice (weighing 23-27 g) were randomly assigned to groups receiving an intraperitoneal injection of i) 100 μL of the vehicle (10 mM Tris pH8.0, 2 mM MgCl2, 4% sucrose; n=17), or ii) 100 μL of vehicle containing 10⁹ pfu of Ad-HO-1 (n=20), or iii) 100 μL of vehicle containing 10⁹ pfu of Ad-β-Gal (n=24), or iv) 100 μL of vehicle containing 10⁹ pfu of Ad-HO-1 and 10⁹ pfu of Ad-β-Gal (n=16). Intraperitoneal injection of adenovirus has been shown to be an effective non-invasive method of administration to target the murine liver (24,25). Specific immune responses, characterized by lymphocyte infiltration, have been reported as early as 4 days following adenovirus administration (4,5). Therefore, in order to ensure observations reflect only acute inflammatory responses, the measurement times were chosen to be within 3 days following infection. Mice were therefore further randomized into different time points: 24 hours, 48 hours (data not shown), or 72 hours. At the designated time, animals underwent intravital video microscopy following which the right lateral hepatic lobe was removed and fixed in 10% buffered formalin and the remaining liver was immediately frozen and stored at −80° C.

Reverse Transcription—Polymerase Chain Reaction Total tissue RNA was extracted by using Trizol reagent (Life Technologies, Rockville, USA), according to the manufacturer's instructions. Primers used for rat HO-1 were sense CTGCTAGCCTGGTTCAAGATA, and antisense CATCTCCTTCCATTCCAGAG. Primers used for β-Galactosidase were sense GACGTCTCGTTGCTGCATAA, and antisense CAGCAGCAGACCATTTTCAA. Primers used for rat glyceraldehyde-3-phosphopate dehydrogenase (GAPDH) were sense TCCCTCAAGATTGTCAGCAA, and antisense AGATCCACAACGGATACATT. The size of the HO-1 product is 316 bp, β-Galactosidase product is 399 bp, and GAPDH product is 309 bp. A reaction mixture (50 μl) was made according to Access RT-PCR System (Promega, Madison, USA), which consisted of 0.8 μg RNA, 10 μl AMV/Tfl 5×reaction buffer, 1 μl dNTP mix (10 mM each dNTP), 50 pmol sense primer, 50 pmol antisense primer, 2 μl 25 mM MgSO4, 1 μl AMV reverse transcriptase (5 U/μl), and 1 μl Tfl DNA polymerase (5 U/μl). Nuclease-free mineral oil (20 μl) was overlaid on the reaction mixture. Conditions for HO-1 and GADPH RT-PCR were 1 cycle at 48° C. for 45 minutes; 1 cycle at 95° C. for 2 minutes; 30 cycles at 95° C. for 30 seconds, 60° C. for 1 minute, and 68° C. for 90 seconds; and 1 cycle at 68° C. for 5 minutes. Conditions for β-Galactosidase were 1 cycle at 48° C. for 45 minutes; 1 cycle at 95° C. for 2 minutes; 36 cycles at 94° C. for 30 seconds, 60° C. for 1 minute, and 68° C. for 1 minute; and 1 cycle at 68° C. for 7 minutes. Each reaction product (10 μl) was then separated on a 1% agarose gel containing 0.5 μg/ml of ethidium bromide. To determine the relative intensity of mRNA bands, they were quantified using an imaging densitometer (BioRad Laboratories, Canada) and expressed as the ratio of HO-1 or β-Galactosidase mRNA to β-actin mRNA.

Histological Analysis Formalin-fixed liver tissue was embedded in paraffin, and 5 μm thick sections were stained with hematoxylin and eosin for histopathological examination. Histopathological analysis of liver sections was performed in a blinded fashion by an experienced hepatopathologist. Cytopathic effects and inflammatory cell infiltration, (specifically, cell ballooning, tissue necrosis, compromised lobular structure, and the presence of minute to large foci of inflammation), were noted. Additionally, randomly selected fields from each liver section were examined and the number of leukocytes (intravascular and extravasated) and macrophages per field of view was counted.

Immunohistochemistry Standard immunohistochemical techniques were used for detection of HO-1. Briefly, serial sections were initially rehydrated and stained with hematoxylin. Endogenous peroxidase activity was inhibited using 3% H₂O₂ in methanol for 5 min. Following 10% horse serum blocking, sections were rinsed with PBS and incubated with 1:100 rabbit anti-HO-1 polyclonal antibody (Stressgen, Victoria, BC, Canada) for 1.5 hr. Following primary incubation, tissue sections were stained for antigen-antibody complexes using a peroxidase detection system (Vectastain ABC kit, Vector Laboratories Inc., Burlingame, Calif.)

Intravital Video Microscopy Intravital video microscopy is an established method for evaluating inflammation (47,48) and provides a unique tool for evaluating the local tissue inflammation resulting from adenovirus administration. Mice were anaesthetized by inhalation of isoflurane (5% induction, 2.5% laparotomy, 2% maintenance) with a mixture of nitrogen (2.5 L/min) and oxygen (1 L/min). A transverse incision was made across the midline just below the xiphoid. The left hepatic lobe was exposed and reflected onto the stage of an inverted microscope (Nikon Eclipse TE300) with a few drops of warmed saline. The liver was covered with plastic film in order to prevent dehydration and minimize movement due to respiration. Throughout microscopy, body temperature was maintained between 36.0° C. and 37.0° C. The liver was illuminated using a fibreoptic light guide and video images were recorded for later analysis. Sufficient magnification was used for easy identification of intravascular leukocytes. Seven fields of view containing a postsinusoidal venule and ten fields of view containing only sinusoids were recorded each for a one-minute observation period. For each venule, the number of leukocytes either rolling or stationary (i.e., exhibiting inflammatory behaviour) over the observation period were counted. These numbers were normalised to the venular area in the field of view and expressed as the number of leukocytes (rolling+stationary) per 100 μm² of venule per minute. For each sinusoidal field of view, the number of leukocytes which remained stationary over the observation period were counted and expressed as the number per field of view.

Alanine Transaminase Prior to euthanasia, a blood sample was obtained by cardiac puncture. Serum levels of Alanine Transaminase (ALT) as an index of hepatocellular injury were determined by standard enzymatic techniques.

Statistics Data are expressed as the mean±the standard error of the mean (SEM). To compare the differences in the mean values among multiple groups, a standard one-way ANOVA followed by post-hoc comparison using the Student-Newman-Keuls test. A p-value of P<0.05 was considered significant.

Results

To evaluate the level of the acute inflammatory response elicited by the adenovirus encoding the gene for heme oxygenase-1 (Ad-HO-1), mice were administered the virus by intraperitoneal injection. Intraperitoneal injection of adenovirus has been shown to be an effective non-invasive method of administration to specifically target the murine liver (24,25). The dose of 10⁹ pfu is commonly utilized and has been shown to initiate significant inflammatory and immune responses (7,17,26-28). Following 24 or 72 hours, the level of hepatic inflammation was quantified using intravital video microscopy and histopathological analysis. This was compared to mice receiving injection of either the vehicle or a control vector containing the gene for β-Galactosidase (Ad-β-Gal). A fourth experimental group received injection of Ad-HO-1 and Ad-β-Gal simultaneously in order to evaluate the effect of Ad-HO-1 on Ad-β-Gal-induced inflammation.

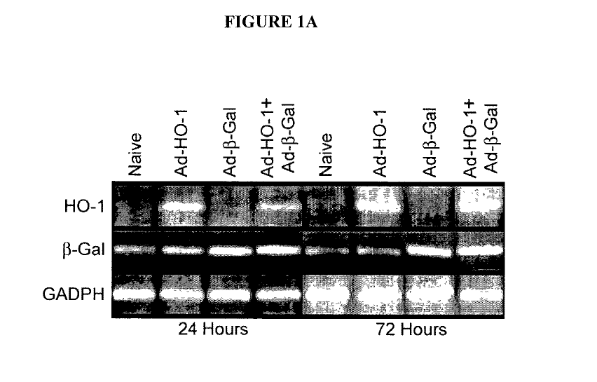

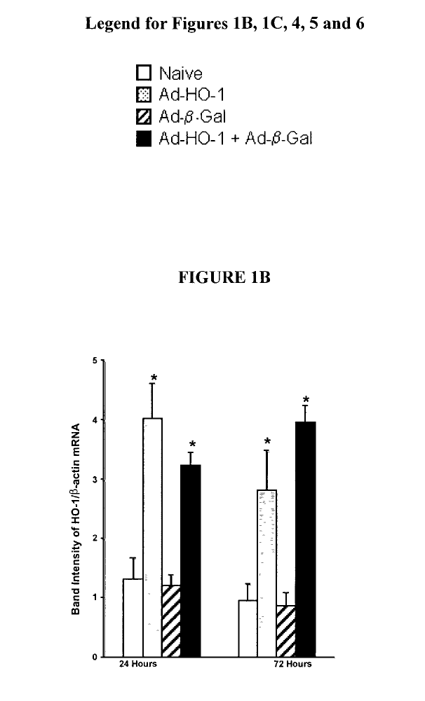

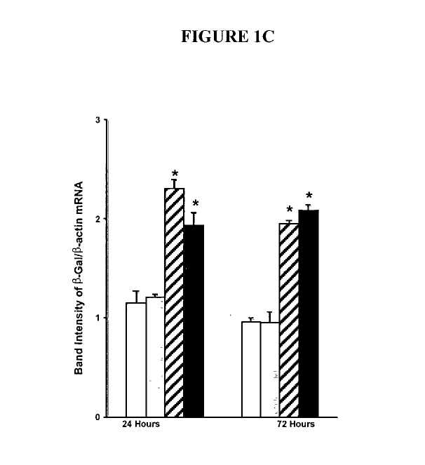

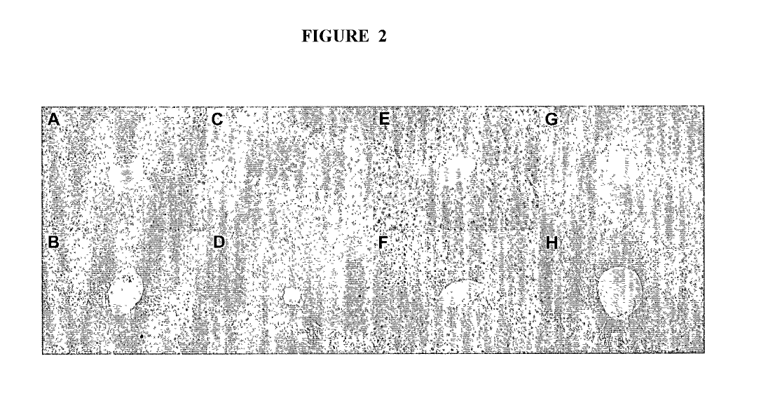

Gene transfer was verified by RT-PCR and immunohistochemistry. Intraperitoneal injection of adenovirus resulted in a significant increase in HO-1 mRNA in liver following injection of Ad-HO-1 and a significant increase in β-Galactosidase following injection of Ad-β-Gal (FIG. 1). Due to the inducible nature of HO-1, it is important to note that Ad-β-Gal administration did not elicit an increase in HO-1 expression. Immunohistochemical staining of HO-1 protein in serial sections (FIG. 2) demonstrated upregulated HO-1 gene expression predominantly in hepatocytes. Endogenous expression of HO-1 in the liver is also shown as the antibody used was cross reactive for mouse HO-1.

Hematoxylin and eosin staining in serial sections (FIG. 2) were examined by an experienced hepatopathologist for cytopathic effects and the infiltration of inflammatory cells. No animals showed viral-hepatitis-like pathological changes (such as cell ballooning, gross tissue necrosis, loss of lobular structure, or presence of large foci of inflammation) throughout the course of the experiment. Generally, animals from all experimental groups showed occasional hepatocyte necrosis and a small number of minute foci of inflammation, consisting predominantly of monocytes. This is consistent with previous studies which demonstrate that severe viral-hepatitus-like changes do not occur until the late phase of adenovirus-induced inflammation (10,26). Serum levels of Alanine Transaminase (ALT) were increased 2-fold 24 hours following injection of Ad-β-Gal (31±6 U/L) compared with both vehicle (14±1 U/L) and Ad-HO-1 administration (15±2 U/L). This increase in ALT was prevented by simultaneous administration of both Ad-β-Gal and Ad-HO-1 (16±2 U/L). There was no increase in ALT measured at 72 hours.

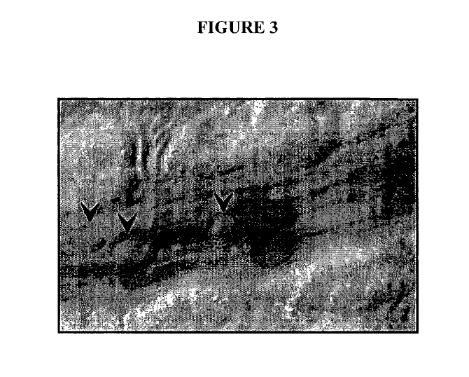

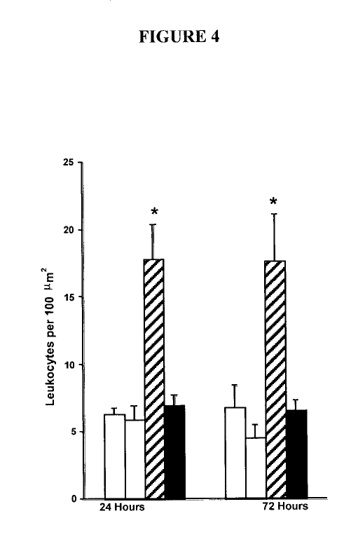

Intravital video microscopy was used to acquire a qualitative in vivo measure of activated intravascular leukocytes (FIG. 3). Leukocytes that were rolling or adhering in postsinusoidal venules were counted over a one-minute observation period (FIG. 4). The total number (rolling+adherent) of leukocytes was significantly elevated (P<0.01) in livers transfected with Ad-β-Gal as compared to vehicle (vehicle 24 hrs=6.3±0.5, 72 hrs=6.8±1.6; Ad-β-Gal 24 hrs=17.8±2.6, 72 hrs=17.6±3.5). There was no observed increase in the number of leukocytes interacting with the wall of postsinusoidal venules in the livers of animals injected with Ad-HO-1 (Ad-HO-1 24 hrs=5.9±1.0, 72 hrs=4.5±1.0). Interestingly, the Ad-β-Gal-induced increase in rolling and adhered leukocytes in postsinusoidal venules was prevented by co-administration of Ad-HO-1 (Ad-HO-1+Ad-β-Gal 24 hrs=7.0±0.7, 72 hrs=6.6±0.7).

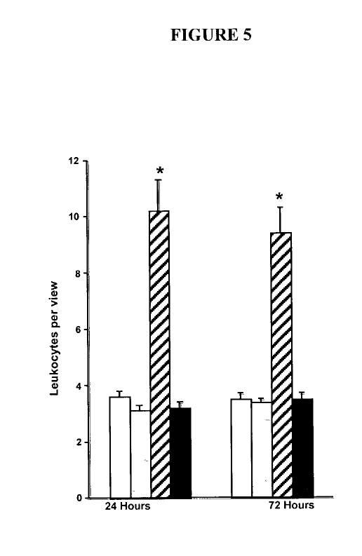

Leukocytes which remained stationary over the one-minute observation period in liver sinusoids were also counted (FIG. 5). The number of stationary leukocytes following injection of Ad-β-Gal was significantly higher (P<0.01) than vehicle (vehicle 24 hrs=3.6±0.2, 72 hrs=3.5±0.2; Ad-β-Gal 24 hrs=10.2±1.1, 72 hrs=9.4±0.9). Ad-HO-1 did not elicit an increase in stationary leukocytes in sinusoids (Ad-HO-1 24 hrs=3.1±0.2, 72 hrs=3.4±0.1). In addition, the Ad-β-Gal-induced increase in stationary leukocytes was prevented by co-administration of Ad-HO-1 (Ad-HO-1+Ad-β-Gal 24 hrs=3.2±0.2, 72 hrs=3.5±0.2).

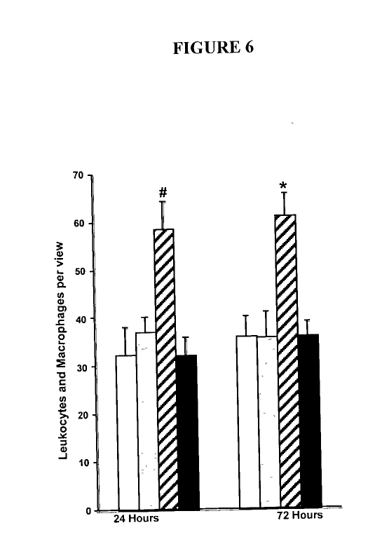

There were notable differences in the number of leukocytes (intravascular and extravasated) and macrophages in hematoxylin and eosin stained liver sections following adenoviral transfection (FIG. 6). There was a significant increase (P<0.05) in the number of leukocytes and macrophages in the livers of Ad-β-Gal injected animals compared with vehicle-injected animals (vehicle 24 hrs=32.5±5.5, 72 hrs=35.9±4.2; Ad-β-Gal 24 hrs=58.4±5.7, 72 hrs=60.9±4.5). Ad-HO-1 did not elicit an increase in leukocytes and macrophages (Ad-HO-1 24 hrs=37.0±3.0, 72 hrs=35.8±5.1). Co-administration of Ad-HO-1 prevented the Ad-β-Gal-induced increase in leukocytes and macrophages (Ad-HO-1+Ad-β-Gal 24 hrs=32.1 ±3.8, 72 hrs=36.0±2.9).

Discussion

Adenovirus vectors hold significant promise for advancing human gene therapy. However, understanding and controlling the immune and inflammatory responses to adenovirus vectors is essential for their effective use in clinical applications. In spite of recent interest, the mechanisms of adenovirus vector-induced acute inflammation remain to be fully elucidated. Acute inflammation is known to be caused both by the transduction of foreign proteins and by the viral capsid. Several studies have demonstrated upregulation of proinflammatory genes and adhesion molecule expression by adenovirus vectors. The C-C chemokine RANTES, interferon-inducible protein-10 (IP-10) (29,30), interleukin-6 (IL-6) (8,31,32), interleukin-8 (IL-8) (16,33), interleukin-12 (IL-12) (31), tumour necrosis factor-α (TNF-α) (8,31,32), and nuclear factor-κB (NF-κB) (8) are induced by adenovirus vectors (13). Intercellular adhesion molecule-1 (ICAM-1) (34,35), vascular cell adhesion molecule-1 (VCAM-1) (35), and adhesion molecule CD34 (35) are upregulated following adenovirus vector administration (34,35). Although the mechanisms of adenovirus-induced acute inflammation require further study, the efficiency of adenovirus administration is known to be greatly improved by adjunct treatment with anti-inflammatory agents (11). This tactic however, may be inappropriate for clinical applications since immune-depletion poses additional risks to the patient.

Carbon monoxide (a vasodilator), ferritin (an antioxidant), and bilirubin (an antioxidant) are produced by heme oxygenase-mediated degradation of heme (18,19). Although heme oxygenase-1 (HO-1) is a known anti-inflammatory, the mechanisms of its anti-inflammatory action have not been completely elucidated. HO activity is known to downregulate ICAM-1 (36,37) and inhibit TNF-α-induced apoptosis (38,39). In addition, carbon monoxide is a potent vasodilator (18), can inhibit platelet aggregation (40), and has been shown to inhibit the proinflammatory cytokines TNF-α, interleukin-1β (IL-1β) and macrophage inflammatory protein-1β (MIP-1β) (41). Bilirubin has been shown to decrease expression of P-selectin (36,42) and E-selectin (42). In spite of early explorations of adenovirus-mediated gene transfer of HO-1, which have demonstrated anti-inflammatory effects consistent with pharmacologic upregulation of HO-1, this is the first study to explore the potential of reducing adenovirus vector-induced acute inflammation using Ad-HO-1.

This study confirmed that Ad-HO-1 does not itself elicit an acute inflammatory response in the liver within 3 days of administration. The control virus Ad-β-Gal, however, induced approximately a 3-fold increase in the number of intravascular leukocytes exhibiting inflammatory behaviour and induced approximately a 2-fold increase in leukocytes and macrophages. When Ad-HO-1 and Ad-β-Gal were administered concurrently, no inflammation was observed either by intravital video microscopy or by histopathological analysis. These data demonstrate that co-administration of Ad-HO-1 prevents the acute inflammation normally caused by Ad-β-Gal. Future studies will focus on the mechanisms involved in the prevention of adenovirus vector-induced acute inflammation by co-administration of Ad-HO-1. These results have a profound implication for gene therapy, since the acute inflammation elicited by an adenovirus vector encoding a therapeutic gene can be prevented by co-administration of Ad-HO-1. The prevention of acute inflammation will greatly improve the safety and efficiency of adenovirus vector administration, providing strong incentive to reconsider the use of adenovirus vectors for human gene therapy.

### EXAMPLE 2

Tables 1-4 summarize data regarding the use of the adenovirus to increase heme oxygenase within the liver. This data proves that the use of the virus was effective in gene transfer over 24 hr and 72 hrs following transfection (Tables 1 and 2 entitled: HO-1 24 hr and 72 hr summary). The use of the adenovirus containing heme oxygenase (ADHO-1) did not cause any increase in other proteins such as β-Galactosidase and using combined treatment with virus containing β-Gal with AdHO-1 did not prevent the increase in liver HO-1 mRNA. Tables 3 and 4 entitled β-Galactosidase mRNA summarizes the data obtained from the use of the adenovirus containing β-Gal. They show that transfection with β-gal did not induced HO-1 mRNA.

In summary, these tables prove that the use of adenoviral transfection to increase HO-1 does not in itself alter the expression of β-gal which was used to induce an inflammatory response. This means that the reduction in the inflammatory response the inventors showed following transfection with AdHO-1 was not the result of altering the inflammatory mediator per se, and thus supports the use of AdHO-1 to reduce the acute inflammatory response caused by other andenoviruses.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
HO-1 mRNA
Summary - 24 hr animals

| | Mean | SEM |
|---|---|---|
| Sham | 1.33 | 0.33 |
| AdHO-1 | 4.03 | 0.58 |
| β-gal | 1.21 | 0.17 |
| β-gal + AdHO-1 | 3.24 | 0.21 |

TABLE 2
Summary - 72 hr animals

| | Mean | SEM |
|---|---|---|
| Sham | 0.95 | 0.28 |
| AdHO-1 | 2.83 | 0.65 |
| β-gal | 0.88 | 0.21 |
| β-gal + AdHO-1 | 3.97 | 0.27 |

TABLE 3
β-GALACTOSIDASE mRNA
Summary - 24 hr animals

| | Mean | SEM |
|---|---|---|
| Sham | 1.16 | 0.11 |
| AdHO-1 | 1.22 | 0.02 |
| β-gal | 2.31 | 0.08 |
| β-gal + AdHO-1 | 1.94 | 0.12 |

TABLE 4
Summary - 72 hr animals

| | Mean | SEM |
|---|---|---|
| Sham | 0.97 | 0.03 |
| AdHO-1 | 0.96 | 0.10 |
| β-gal | 1.96 | 0.02 |
| β-gal + AdHO-1 | 2.09 | 0.05 |

### REFERENCES

1. Crystal R G. Transfer of genes to humans: early lessons and obstacles to success. Science 1995; %20;270(5235):404-410.

2. Wilson J M. Adenoviruses as gene-delivery vehicles. N Engl J Med 1996; 334(18):1185-1187.

3. Simon R H, Engelhardt J F, Yang Y, Zepeda M, Weber-Pendleton S, Grossman M et al. Adenovirus-mediated transfer of the CFTR gene to lung of nonhuman primates: toxicity study. Hum Gene Ther 1993; 4(6):771-780.

4. Prince G A, Porter D D, Jenson A B, Horswood R L, Chanock R M, Ginsberg H S. Pathogenesis of adenovirus type 5 pneumonia in cotton rats (Sigmodon hispidus). J Virol 1993; 67(1):101-111.

5. Yei S, Mittereder N, Wert S, Whitsett J A, Wilmott R W, Trapnell B C. In vivo evaluation of the safety of adenovirus-mediated transfer of the human cystic fibrosis transmembrane conductance regulator cDNA to the lung. Hum Gene Ther 1994; 5(6):731-744.

6. Zsengeller Z K, Wert S E, Hull W M, Hu X, Yei S, Trapnell B C et al. Persistence of replication-deficient adenovirus-mediated gene transfer in lungs of immune-deficient (nu/nu) mice. Hum Gene Ther 1995; 6(4):457-467.

7. Adesanya M R, Redman R S, Baum B J, O'Connell B C. Immediate inflammatory responses to adenovirus-mediated gene transfer in rat salivary glands. Hum Gene Ther 1996; 7(9): 1085-1093.

8. Lieber A, He C Y, Meuse L, Schowalter D, Kirillova I, Winther B et al. The role of Kupffer cell activation and viral gene expression in early liver toxicity after infusion of recombinant adenovirus vectors. J Virol 1997; 71(11):8798-8807.

9. Yang Y, Nunes F A, Berencsi K, Furth E E, Gonczol E, Wilson J M. Cellular immunity to viral antigens limits E1-deleted adenoviruses for gene therapy. Proc Natl Acad Sci USA 1994; 91(10):4407-4411.

10. Yang Y, Li Q, Ertl H C, Wilson J M. Cellular and humoral immune responses to viral antigens create barriers to lung-directed gene therapy with recombinant adenoviruses. J Virol 1995; 69(4):2004-2015.

11. Otake K, Ennist D L, Harrod K, Trapnell B C. Nonspecific inflammation inhibits adenovirus-mediated pulmonary gene transfer and expression independent of specific acquired immune responses. Hum Gene Ther 1998; 9(15):2207-2222.

12. Worgall S, Wolff G, Falck-Pedersen E, Crystal R G. Innate immune mechanisms dominate elimination of adenoviral vectors following in vivo administration. Hum Gene Ther 1997; 8(1):37-44.

13. Muruve D A, Barnes M J, Stillman I E, Libermann T A. Adenoviral gene therapy leads to rapid induction of multiple chemokines and acute neutrophil-dependent hepatic injury in vivo. Hum Gene Ther 1999; 10(6):965-976.

14. Somia N, Verma I M. Gene therapy: trials and tribulations. Nat Rev Genet 2000; 1(2):91-99.

15. Stolberg S G. The biotech death of Jesse Gelsinger. N Y Times Mag 1999;:136-140, 149-150.:136-150.

16. Amin R, Wilmott R, Schwarz Y, Trapnell B, Stark J. Replication-deficient adenovirus induces expression of interleukin-8 by airway epithelial cells in vitro. Hum Gene Ther 1995; 6(2):145-153.

17. Christ M, Lusky M, Stoeckel F, Dreyer D, Dieterle A, Michou A I et al. Gene therapy with recombinant adenovirus vectors: evaluation of the host immune response. Immunol Lett 1997; 57(1-3):19-25.

18. Maines M D. The heme oxygenase system: a regulator of second messenger gases. Annu Rev Pharmacol Toxicol 1997; 37:517-54.:517-554.

19. Otterbein L E, Choi A M. Heme oxygenase: colors of defense against cellular stress. Am J Physiol Lung Cell Mol Physiol 2000; 279(6):L1029-L1037.

20. Abraham N G, da Silva J L, Dunn M W, Kigasawa K, Shibahara S. Retinal pigment epithelial cell-based gene therapy against hemoglobin toxicity. Int J Mol Med 1998; 1(4):657-663.

21. Abraham N G, da Silva J L, Lavrovsky Y, Stoltz R A, Kappas A, Dunn M W et al. Adenovirus-mediated heme oxygenase-1 gene transfer into rabbit ocular tissues. Invest Ophthalmol Vis Sci 1995; 36(11):2202-2210.

22. Otterbein L E, Kolls J K, Mantell L L, Cook J L, Alam J, Choi A M. Exogenous administration of heme oxygenase-1 by gene transfer provides protection against hyperoxia-induced lung injury. J Clin Invest 1999; 103(7):1047-1054.

23. Amersi F, Buelow R, Kato H, Ke B, Coito A J, Shen X D et al. Upregulation of heme oxygenase-1 protects genetically fat Zucker rat livers from ischemia/reperfusion injury. J Clin Invest 1999; 104(11):1631-1639.

24. Huard J, Lochmuller H, Acsadi G, Jani A, Massie B, Karpati G. The route of administration is a major determinant of the transduction efficiency of rat tissues by adenoviral recombinants. Gene Ther 1995; 2(2):107-115.

25. Kass-Eisler A, Falck-Pedersen E, Elfenbein D H, Alvira M, Buttrick P M, Leinwand L A. The impact of developmental stage, route of administration and the immune system on adenovirus-mediated gene transfer. Gene Ther 1994; 1(6):395-402.

26. Wang Q, Greenburg G, Bunch D, Farson D, Finer M H. Persistent transgene expression in mouse liver following in vivo gene transfer with a delta E1/delta E4 adenovirus vector. Gene Ther 1997; 4(5):393-400.

27. Nakatani T, Kuriyama S, Tominaga K, Tsujimoto T, Mitoro A, Yamazaki M et al. Assessment of efficiency and safety of adenovirus mediated gene transfer into normal and damaged murine livers. Gut 2000;

47(4):563-570.

28. Dedieu J F, Vigne E, Torrent C, Jullien C, Mahfouz I, Caillaud J M et al. Long-term gene delivery into the livers of immunocompetent mice with E1/E4-defective adenoviruses. J Virol 1997; 71(6):4626-4637.

29. Borgland S L, Bowen G P, Wong N C, Libermann T A, Muruve D A. Adenovirus vector-induced expression of the C-X-C chemokine IP-10 is mediated through capsid-dependent activation of NF-kappaB. J Virol 2000; 74(9):3941-3947.

30. Bowen G P, Borgland S L, Lam M, Libermann T A, Wong N C, Muruve D A. Adenovirus vector-induced inflammation: capsid-dependent induction of the C-C chemokine RANTES requires NF-kappa B. Hum Gene Ther 2002; 13(3):367-379.

31. Zhang Y, Chirmule N, Gao G P, Qian R, Croyle M, Joshi B et al. Acute cytokine response to systemic adenoviral vectors in mice is mediated by dendritic cells and macrophages. Mol Ther 2001; 3(5 Pt 1):697-707.

32. Zsengeller Z, Otake K, Hossain S A, Berclaz P Y, Trapnell B C. Internalization of adenovirus by alveolar macrophages initiates early proinflammatory signaling during acute respiratory tract infection. J Virol 2000; 74(20):9655-9667.

33. Bruder J T, Kovesdi I. Adenovirus infection stimulates the Raf/MAPK signaling pathway and induces interleukin-8 expression. J Virol 1997; 71(1):398-404.

34. Stark J M, Amin R S, Trapnell B C. Infection of A549 cells with a recombinant adenovirus vector induces ICAM-1 expression and increased CD-18-dependent adhesion of activated neutrophils. Hum Gene Ther 1996; 7(14):1669-1681.

35. Rafii S, Dias S, Meeus S, Hattori K, Ramachandran R, Feuerback F et al. Infection of endothelium with E1(−)E4(+), but not E1(−)E4(−), adenovirus gene transfer vectors enhances leukocyte adhesion and migration by modulation of ICAM-1, VCAM-1, CD34, and chemokine expression. Circ Res 2001; 88(9):903-910.

36. Wagener F A, Eggert A, Boerman O C, Oyen W J, Verhofstad A, Abraham N G et al. Heme is a potent inducer of inflammation in mice and is counteracted by heme oxygenase. Blood 2001; 98(6):1802-1811.

37. Wagener F A, da Silva J L, Farley T, de Witte T, Kappas A, Abraham N G. Differential effects of heme oxygenase isoforms on heme mediation of endothelial intracellular adhesion molecule 1 expression. J Pharmacol Exp Ther 1999; 291(1):416-423.

38. Petrache I, Otterbein L E, Alam J, Wiegand G W, Choi A M. Heme oxygenase-1 inhibits TNF-alpha-induced apoptosis in cultured fibroblasts. Am J Physiol Lung Cell Mol Physiol 2000; 278(2):L312-L319.

39. Silver B J, Hamilton B D, Toossi Z. Suppression of TNF-alpha gene expression by hemin: implications for the role of iron homeostasis in host inflammatory responses. J Leukoc Biol 1997; 62(4):547-552.

40. Wagner C T, Durante W, Christodoulides N, Hellums J D, Schafer A I. Hemodynamic forces induce the expression of heme oxygenase in cultured vascular smooth muscle cells. J Clin Invest 1997; 100(3):589-596.

41. Otterbein L E, Bach F H, Alam J, Soares M, Tao L H, Wysk M et al. Carbon monoxide has anti-inflammatory effects involving the mitogen-activated protein kinase pathway. Nat Med 2000; 6(4):422-428.

42. Vachharajani T J, Work J, Issekutz A C, Granger D N. Heme oxygenase modulates selectin expression in different regional vascular beds. Am J Physiol Heart Circ Physiol 2000; 278(5):H1613-H1617.

43. Shibahara S, Muller R, Taguchi H, Yoshida T. Cloning and expression of cDNA for rat heme oxygenase. Proc Natl Acad Sci USA 1985; 82(23):7865-7869.

44. Graham F L, Prevec L. Methods for construction of adenovirus vectors. Mol Biotechnol 1995; 3(3):207-220.

45. Kolls J, Peppel K, Silva M, Beutler B. Prolonged and effective blockade of tumor necrosis factor activity through adenovirus-mediated gene transfer. Proc Natl Acad Sci USA 1994; 91(1):215-219.

46. Ye X, Robinson M B, Batshaw M L, Furth E E, Smith I, Wilson J M. Prolonged metabolic correction in adult ornithine transcarbamylase-deficient mice with adenoviral vectors. J Biol Chem 1996; 271(7):3639-3646.

47. Ferguson D, McDonagh P F, Biewer J, Paidas C N, Clemens M G. Spatial relationship between leukocyte accumulation and microvascular injury during reperfusion following hepatic ischemia. Int J Microcirc Clin Exp 1993; 12(1):45-60.

48. Uhlmann S, Uhlmann D, Spiegel H U. Evaluation of hepatic microcirculation by in vivo microscopy. J Invest Surg 1999; 12(4):179-193.

49. McCoubrey W K Jr, Huang T J, Maines M D. Isolation and characterization of a cDNA from rat brain that encodes hemoprotein heme oxygenase-3. Eur. J Biochem 24(2): 725-732, 1997.

50. McCoubrey W K Jr, Maines M D. The structure, organization and differential expression of the gene encoding rat heme oxygenase-2. Gene 139(2): 155-161, 1994.

51. Alam J, Cai J, Smith A. Isolation and characterization of the mouse heme oxygenase-1 gene. Distal 5′ sequences are required for induction by heme or heavy metals. J Biol Chem 269(2): 1001-1009, 1994.

52. Shibahara S, Muller R, Taguchi H and Yoshida T. Proc. Natl. Acad Sci USA 82:7865-7869, 1985.

We claim:

1. A method of reducing an immune response to a first nucleic acid construct comprising administering an effective amount of a second nucleic acid construct comprising a nucleic acid sequence encoding an anti-inflammatory protein to a cell or animal in need thereof.

2. A method according to claim 1 wherein the anti-inflammatory protein is a heme oxygenase.

3. A method according to claim 1 wherein the second nucleic acid construct is in a viral vector.

4. A method according to claim 3 wherein the viral vector is an adenovirus.

5. A method according to claim 1 wherein the first nucleic acid construct is in a viral vector.

6. A method according to claim 5 wherein the viral vector is an adenovirus.

7. A method according to claim 1 wherein the immune response that is reduced is an inflammatory response.

8. A method according to claim 7 wherein the inflammatory response is within the liver.

9. A method according to claim 1 wherein the anti-inflammatory protein is selected from the group consisting of super oxide dismutase, catalase, glutathione, nitric oxide synthase, heat shock protein-70, interleukin-4 and interleukin-10.

10. A method according to claim 1 wherein the first nucleic acid construct contains a heterologous nucleic acid seqeuence.

11. A method according to claim 10 wherein the heterologous nucleic acid sequence encodes a therapeutic, diagnostic or prophylactic protein.

12. A method according to claim 10 wherein the heterologous nucleic acid sequence encodes a therapeutic protein.

13. A method according to claim 7 for reducing an inflammatory response to a vector used in gene therapy comprising administering (i) a first vector comprising a heterologous nucleic acid sequence and (ii) a second vector comprising a nucleic acid sequence encoding an anti-inflammatory protein to an animal in need thereof.

14. A method according to claim 13 wherein the vector is a viral vector.

15. A method according to claim 14 wherein the viral vector is an adenovirus.

16. A method according to claim 13 wherein the heterologous nucleic acid sequence encodes a protein that is used to treat liver disease, cancer, transplant rejection, cystic fibrosis, nervous system disorders or genetic disorders.

17. A method according to claim 13 wherein the anti-inflammatory protein is a heme oxygenase.

18. A method according to claim 13 wherein the anti-inflammatory protein is selected from the group consisting of super oxide dismutase, catalase, glutathione, nitric oxide synthase, heat shock protein-70, interleukin-4 and interleukin-10.

19. A method according to claim 7 for reducing an inflammatory response to a vector used in gene therapy comprising administering a vector for gene therapy comprising a heterologous nucleic acid sequence and a nucleic acid sequence encoding an anti-inflammatory protein to an animal in need thereof.

20. A method according to claim 19 wherein the vector is a viral vector.

21. A method according to claim 20 wherein the viral vector is an adenovirus.

22. A method according to claim 19 wherein the anti-inflammatory protein is a heme oxygenase.

23. A method according to claim 19 wherein the anti-inflammatory protein is selected from the group consisting of super oxide dismutase, catalase, glutathione, nitric oxide synthase, heat shock protein-70, interleukin-4 and interleukin-10.

24. A method according to claim 19 wherein the heterologous nucleic acid sequence encodes a protein that is used to treat liver disease, cancer, transplant rejection, cystic fibrosis, nervous system disorders or genetic disorders.
